(19) United States
(12) Patent Application Publication
(10) Pub. No.: US 2016/0251626 A1
(43) Pub. Date: Sep. 1, 2016

(54) THREE-DIMENSIONAL TISSUE AND PRODUCTION METHOD THEREFOR

(71) Applicants: OSAKA UNIVERSITY, Suita-shi, Osaka (JP); PUBLIC UNIVERSITY CORPORATION YOKOHAMA CITY UNIVERSITY, Yokohama-shi, Kanagawa (JP)

(72) Inventors: Mitsuru Akashi, Suita-shi, Osaka (JP); Michiya Matsusaki, Suita-shi, Osaka (JP); Yoshihiro Ishikawa, Yokohama-shi, Kanagawa (JP); Utako Yokoyama, Yokohama-shi, Kanagawa (JP)

(73) Assignees: OSAKA UNIVERSITY, Suita-shi, Osaka (JP); PUBLIC UNIVERSITY CORPORATION YOKOHAMA City UNIVER SITY, Yokohama-shi, Kanagawa (JP)

(21) Appl. No.: 15/028,204

(22) PCT Filed: Oct. 9, 2014
(86) PCT No.: PCT/JP2014/077083

§ 371 Date: Apr. 13, 2016

(30) Foreign Application Priority Data
Oct. 10, 2013 (JP) 2013-212966

(51) Int. Cl. C12N 5/077

(52) U.S. Cl. CPC C12N 5/0661

(57) ABSTRACT

Provided are an elastic three-dimensional tissue and a method by which the tissue can be produced. The elastic three-dimensional tissue includes smooth muscle cells and an extracellular matrix component, with the smooth muscle cells being layered with the extracellular matrix component interposed therebetween. Furthermore, the production method for a three-dimensional tissue includes layering smooth muscle cells with an extracellular matrix component interposed therebetween, wherein the smooth muscle cells are those directed towards a differentiated type from an undifferentiated type.

### TECHNICAL FIELD

The present disclosure relates to a three-dimensional tissue and a production method therefor.

### BACKGROUND ART

Vascular models with three-dimensionalized vascular smooth muscle cells have been called for widely in terms of surgical treatments for vascular diseases such as injuries and arteriosclerosis as well as in terms of providing new alternatives to animal experiments, the review of which has been required in recent years. Therefore, researches have been conducted for artificially three-dimensionalizing vascular smooth muscle cells to establish vascular models (for example, Non-Patent Documents 1 and 2). Non-Patent Document 1 discloses an artificial vascular model obtained as follows: after smooth muscle cells were cultured in a culture medium containing highly expressed collagen for a few weeks and then were dissociated, they were rolled into a blood vessel shape and further were cultured for a few weeks. Furthermore, Non-Patent Document 2 discloses that rat neonate vascular smooth muscle cells and human umbilical vein vascular smooth muscle cells are layered using a technique for layering cells by forming fibronectin and gelatin nano thin films disclosed in Patent Document 1 and thereby a layered product of smooth muscle cells similar to a vascular wall is formed.

### PRIOR ART DOCUMENTS

### Patent Document

[Patent Document 1] JP 4919464 B

### Non-Patent Documents

[Non-Patent Document 1] L'Heureux et al., FASEB J. 12, 47 (1998)

[Non-Patent Document 2] M. Matsusaki et al., Journal of Biomaterials Science 23 (2012) 63-79

### DISCLOSURE OF INVENTION

### Problem to be Solved by the Invention

For the purpose of transplant, various vascular prostheses have been proposed. However, although vascular medial layers, particularly arterial medial layers are elongatable and elastic, elastic vascular prostheses have not been proposed yet. For example, Non-Patent Document 1 describes that vascular prostheses obtained therein are rigid but does not describe that they are elastic. Furthermore, the method of Non-Patent Document 2 has a problem that the layered product obtained therein has a low expression of elastic fibers and thereby is less self-supporting after being dissociated from a base material. Furthermore, the method of Non-Patent Document 1 has a problem that it takes a few months to produce a transplantable vascular model.

With the above in mind, the present disclosure provides an elastic three-dimensional tissue and a method by which the tissue can be produced.

### Means for Solving Problem

In one or more of embodiments, the present disclosure relates to an elastic three-dimensional tissue including smooth muscle cells and an extracellular matrix component, with the smooth muscle cells being layered with the extracellular matrix component interposed therebetween.

In one or more of embodiments, the present disclosure relates to a production method for a three-dimensional tissue including layering smooth muscle cells with an extracellular matrix component interposed therebetween, wherein the smooth muscle cells are those directed towards a differentiated type from an undifferentiated type.

### Effects of the Invention

According to the present disclosure, a three-dimensional tissue containing elastic fibers can be provided in one or more of embodiments.

### BRIEF DESCRIPTION OF DRAWINGS

FIG. 1A in FIG. 1 shows an image of the three-dimensional tissue of Example 1. B in FIG. 1 shows an image of the blood vessel of a rat neonate, C in FIG. 1 shows an image of the blood vessel of an adult rat, and D in FIG. 1 shows an image of the three-dimensional tissue of Comparative Example 1.

FIG. 2 shows examples of photographs of the tissue sections subjected to fluorescent immunostaining of the three-dimensional tissue of Example 1 and the blood vessel of a rat neonate.

FIG. 3 shows examples of images of an experiment for evaluating the elasticity of the three-dimensional tissue of Example 1.

### DESCRIPTION OF THE INVENTION

The present disclosure is based on the knowledge that smooth muscle cells directed towards a differentiated type from an undifferentiated type are layered with an extracellular matrix component interposed therebetween to give a three-dimensional tissue and thereby an elastic three-dimensional tissue can be produced.

The mechanism, in which smooth muscle cells directed towards a differentiated type from an undifferentiated type are layered with an extracellular matrix component interposed therebetween and thereby an elastic three-dimensional tissue can be produced, is not clear but is inferred as follows. That is, even after the smooth muscle cells directed towards a differentiated type from an undifferentiated type are treated with a cell dissociation reagent such as trypsin for cell recovery the characteristics of the smooth muscle cells are maintained. Furthermore, it is considered that when the smooth muscle cells directed towards a differentiated type from an undifferentiated type are layered and then are cultured, said smooth muscle cells thus layered secrete an extracellular matrix component in the three-dimensional tissue, the extracellular matrix component thus secreted contributes to the expression of elastic fibers, and thereby an elastic three-dimensional tissue is obtained. However, the present disclosure does not need to be interpreted to be limited to this mechanism.

In the present disclosure, the “smooth muscle cells directed towards a differentiated type from an undifferentiated type” include, in one or more of embodiments, smooth muscle cells that exhibit differentiated characteristics as well as smooth muscle cells with both differentiated characteristics and undifferentiated characteristics (that is, smooth muscle cells in the process of differentiation from an undifferentiated type to a differentiated type). In one or more of embodiments, the smooth muscle cells of a differentiated type (a contractile type) are smooth muscle cells that are rich in contractile protein, specialized in contraction, and/or low in division capacity (proliferative capacity) as compared to smooth muscle cells of undifferentiated type (a synthetic type). In one or more of embodiments, whether the smooth muscle cells are “smooth muscle cells directed towards a differentiated type from an undifferentiated type” or not can be determined by culturing smooth muscle cells for one, two, three, four, or five days and then checking the level of proliferative capacity. Furthermore, it also can be determined using markers such as SM22, SM1, SM2, and SMemb. In the cells directed towards a differentiated type, SM22, SM1, and SM2 are highly expressed while the expression of SMemb is reduced, as compared to cells of an undifferentiated type.

In one or more of embodiments, smooth muscle cells directed towards a differentiated type from an undifferentiated type can be obtained through differentiation of smooth muscle cells or through transformation of smooth muscle cells, and preferably, they can be obtained through transformation of smooth muscle progenitor cells or smooth muscle cells of an undifferentiated or dedifferentiated type into smooth muscle cells of a differentiated type (a contractile type). In one or more of embodiments, transformation can be achieved by culturing smooth muscle cells at a high density. In the present disclosure, “culturing smooth muscle cells at a high density” denotes culturing smooth muscle cells in a substantially 100% confluent state. In one or more of embodiments, the phrase “substantially 100% confluent” includes at least 95%, at least 96%, at least 97%, at least 98%, at least 99%, or 100% confluent. The smooth muscle cells subcultured under general culture conditions are normally smooth muscle cells of an undifferentiated type with proliferative capacity. Examples of general culture conditions include culturing at not more than 80%, not more than 70%, or not more than 50% confluent.

In the present disclosure, “smooth muscle cells” denote cells that compose or can compose smooth muscle. In one or more of embodiments, examples of smooth muscle cells include vascular smooth muscle cells and tracheal smooth muscle cells. The origin of smooth muscle cells is not particularly limited but in one or more of embodiments, examples of the origin include human beings and animals other than human beings. The animals other than human beings are not particularly limited but examples thereof include primates (for example, Macaca mulatta), mice, rats, dogs, rabbits, and pigs. Human beings are preferred in terms of allowing smooth muscle cells to exhibit characteristics and functions that are equivalent to those of a human biological tissue as much as possible. Furthermore, they may be smooth muscle cells obtained through induction of differentiation of embryonic stein cells (ES cells), human mesenchymal stem cells (MSC), or induced pluripotent stem cells (iPS cells).

In the present disclosure, “elastic” denotes the property that a load applied to a three-dimensional tissue results in elongation of the three-dimensional tissue and unloading allows it to return to approximately the original size. In one or more of embodiments, examples of the load applied to the three-dimensional tissue include tensile force. In the present disclosure, “elastic” denotes in one or more of embodiments that the three-dimensional tissue can be elongated to at least 1.2 times its length, preferably at least 1.3 times, 1.4 times, 1.5 times, or twice its length, and more preferably that after being elongated, the three-dimensional tissue can return to its original size. In the present disclosure, the phrase “can be elongated to at least 1.2 times its length” denotes that with the length of the three-dimensional tissue before being elongated in the direction of elongation being taken as 1, the length of the three-dimensional tissue in the direction of elongation after being elongated is 1.2 or longer. Furthermore, in one or more of embodiments, “elastic” in the present disclosure denotes a high expression of elastic fibers in the three-dimensional tissue.

In one or more of embodiments, the expression of elastic fibers can be evaluated by Elastica van Gieson stain or radioisotope ([³H]valine).

In the present disclosure, the phrase “smooth muscle cells are layered with an extracellular matrix component interposed therebetween” denotes that smooth muscle cells are three-dimensionally stacked together, with an extracellular matrix component being interposed therebetween, and preferably that a plurality of cell layers containing smooth muscle cells are layered. In one or more of embodiments, the phrase “a plurality of cell layers are layered” denotes that the cell culture product is not that in which the cell layer is a monolayer.

In the present disclosure, an “extracellular matrix component” denotes a substance that serves functions such as a mechanical supportive function provided by filling the space outside cells in a biological body, a function for providing a scaffold, and/or a function for maintaining the biological factor. Furthermore, the extracellular matrix component may further contain a substance that can serve functions such as a skeletal function, a function for providing a scaffold, and/or a function for maintaining the biological factor in in vitro cell culture. The extracellular matrix component may also contain a substance produced artificially or a part thereof. The extracellular matrix components that can be used include the examples described later or those disclosed in JP 4919464 B and JP 2012-115254A.

In the present disclosure, a “three-dimensional tissue” denotes that which includes an extracellular matrix component and smooth muscle cells layered with the extracellular matrix component interposed therebetween and which is elastic. In one or more of embodiments, it can be checked by detecting alpha SMA (smooth muscle actin) as positive that the cells contained in the three-dimensional tissue are smooth muscle cells. In one or more of embodiments, the three-dimensional tissue of the present disclosure may contain cells other than smooth muscle cells. In one or more of embodiments, examples of the cells other than smooth muscle cells include vascular endothelial cells, fibroblasts, and hemocyte-derived cells. The origin of the cells contained in the three-dimensional tissue of the present disclosure is not particularly limited but in one or more of embodiments, examples of the origin thereof include human beings and animals other than human beings. The animals other than human beings are as described above.

### Three-Dimensional Tissue

In one or more of embodiments, the present disclosure relates to an elastic three-dimensional tissue including smooth muscle cells and an extracellular matrix component, with the smooth muscle cells being layered with the extracellular matrix component interposed therebetween (hereinafter also referred to as a “three-dimensional tissue of the present disclosure”). Since the three-dimensional tissue of the present disclosure is elastic and, in one or more of embodiments, contains elastic fibers expressed at a high level, it can be used as a tissue fragment that exhibits an excellent self-supporting property, i.e., maintains the three-dimensional structure without a supporter. In one or more of embodiments, therefore, the three-dimensional tissue of the present disclosure can be formed into, for example, a tubular shape. The three-dimensional tissue of the present disclosure can be produced by a production method of the present disclosure described later.

In one or more of embodiments, the three-dimensional tissue of the present disclosure includes a medial layer containing an extracellular matrix component and layered smooth muscle cells as well as an intimal layer containing endothelial cells formed on the medial layer. In one or more of embodiments, the three-dimensional tissue of the present disclosure includes an adventitial layer, a medial layer formed on the adventitial layer, and an intimal layer formed on the medial layer, wherein the adventitial layer contains fibroblasts, the medial layer contains an extracellular matrix component and layered smooth muscle cells, and the intimal layer contains endothelial cells.

The three-dimensional tissue of the present disclosure has an excellent self-supporting property, which, in one or more of embodiments, allows it to be used as a blood vessel for transplant and to be formed as a vascular prosthesis. Since the three-dimensional tissue of the present disclosure is elastic, it can also be used as a vascular prosthesis for a blood vessel having a small diameter and bends such as a coronary artery in one or more of embodiments. Furthermore, since the three-dimensional tissue of the present disclosure is elastic as is the case with a blood vessel in a biological body, it can also be used as a vascular model for elucidating molecular mechanisms of vascular diseases or evaluating pharmacological effects in one or more of embodiments.

### Production Method for Three-Dimensional Tissue

In one or more of embodiments, the present disclosure relates to a production method for a three-dimensional tissue including layering smooth muscle cells with an extracellular matrix component interposed therebetween, wherein the smooth muscle cells are those directed towards a differentiated type from an undifferentiated type (hereinafter, also referred to as a “production method of the present disclosure”). According to the production method of the present disclosure, in one or more of embodiments, a three-dimensional tissue with a high expression of elastic fibers and an excellent self-supporting property can be produced in a short period of one week to a few weeks after the start of layering cells.

In one or more of embodiments, layering the smooth muscle cells with the extracellular matrix component interposed therebetween include layering the smooth muscle cells using a cell suspension containing cells directed towards a differentiated type from an undifferentiated type.

In one or more of embodiments, the production method of the present disclosure may include preparing a cell suspension. In one or more of embodiments, the cell suspension can be prepared by dispersing smooth muscle cells directed towards a differentiated type from an undifferentiated type in, for example, a culture medium. In one or more of embodiments, in terms of differentiating the smooth muscle cells into a differentiated type, the preparation of the cell suspension includes culturing smooth muscle cells at a high density. The period of culturing at a high density can be determined suitably according to the origin of the smooth muscle cells. In the case of smooth muscle cells derived from a rat or a mouse, the period of culturing at a high density is, in one or more of embodiments, at least six days, at least seven days, or at least eight days, or not more than 20 days or not more than 15 days. Furthermore, in the case of smooth muscle cells derived from a human being, the period of culturing at a high density is, in one or more of embodiments, at least two days but not more than ten days, not more than eight days, or not more than five days. The culture temperature is not particularly limited but is 4 to 60° C., 20 to 40° C., or 30 to 37° C. in one or more of embodiments. In one or more of embodiments, examples of the culture medium include Eagle's MEM culture medium, Dulbecco's Modified Eagle culture medium (DMEM), Modified Eagle culture medium (MEM), Minimum Essential culture medium, RDMI, and GlutaMax culture medium. In one or more of embodiments, the culture medium may be a serum-added culture medium or may be a serum-free culture medium.

In terms of increasing the amount of elastic fibers produced in the three-dimensional tissue and improving the elasticity of the three-dimensional tissue, in one or more of embodiments, examples of the smooth muscle cells to be cultured at a high density include smooth muscle progenitor cells and smooth muscle cells of a synthetic type as well as smooth muscle cells in fetal period or smooth muscle cells up to infancy. It has been known that in one or more of embodiments, the smooth muscle cells up to infancy have a high proliferative capacity, produce, for example, extracellular matrices and growth factors actively, and are of a synthetic type. In one or more of embodiments, the smooth muscle cells can be taken from, for example, an artery. Examples of the artery include an aorta, a coronary artery a pulmonary artery, and an umbilical artery. In one or more of embodiments, the smooth muscle cells up to infancy can be taken from, for example, an umbilical artery.

In one or more of embodiments, the preparation of the cell suspension includes a treatment for dissociating cells cultured at a high density. In one or more of embodiments, examples of the cell dissociation reagent that is used for the dissociation treatment include trypsin. The conditions for the dissociation treatment are not particularly limited. The temperature for the dissociation treatment is not particularly limited but is 4 to 60° C., 20 to 40° C., or 30 to 37° C. in one or more of embodiments. The dissociation treatment time is not particularly limited but is 10 to 120 minutes, 15 to 60 minutes, or 15 to 45 minutes in one or more of embodiments. In one or more of embodiments, the preparation of the cell suspension includes dispersing the cells subjected to the dissociation treatment in a culture medium. The culture medium is as described above.

In one or more of embodiments, layering smooth muscle cells with an extracellular matrix component interposed therebetween can be carried out by alternately carrying out the formation of a cell layer containing smooth muscle cells directed towards a differentiated type from an undifferentiated type (hereinafter also referred to simply as a “cell layer”) and the formation of a layer containing an extracellular matrix component (hereinafter also referred to as an “extracellular matrix component layer”) (a first layering method) or by layering smooth muscle cells directed towards a differentiated type from an undifferentiated type that are coated with an extracellular matrix component (a second layering method).

### First Layering Method

The first layering method includes alternately carrying out the formation of a cell layer and the formation of an extracellular matrix component layer to layer a plurality of cell layers containing smooth muscle cells directed towards a differentiated type from an undifferentiated type. In one or more of embodiments, the formation of a cell layer can be carried out by placing a cell suspension containing smooth muscle cells directed towards a differentiated type from an undifferentiated type on a base material or an extracellular matrix component layer and culturing it. In one or more of embodiments, the density of the smooth muscle cells directed towards a differentiated type from an undifferentiated type in the cell suspension is 1×10² to 1×10⁷ pcs/mL, 1×10³ to 1×10⁶ pcs/mL, or 1×10³ to 1×10⁵ pcs/mL. In one or more of embodiments, the density of the smooth muscle cells directed towards a differentiated type from an undifferentiated type to be placed is 1×10² to 1×10⁹ pcs/cm², 1×10⁴ to 1×10⁸ pcs/cm², 1×10⁵ to 1×10⁷ pcs/cm², or 1×10⁵ to 1×10⁶ pcs/cm². In one or more of embodiments, the incubation temperature is 4 to 60° C., 20 to 40° C., or 30 to 37° C. In one or more of embodiments, the incubation time per formation of one cell layer is 1 to 24 hours, 3 to 12 hours, or 3 to 6 hours. The base material is not particularly limited, and those conventionally known and those to be developed from now on can be used.

In one or more of embodiments, the extracellular matrix component layer can be formed by placing a solution containing an extracellular matrix component on a cell layer. In one or more of embodiments, the extracellular matrix component layer can be formed by alternately placing a solution containing a substance A (solution A) and a solution containing a substance B that interacts with the substance A (solution B) on a cell layer. In one or more of embodiments, it is preferable that with an alternate placement of the solution A and the solution B being taken as one set, the extra cellular matrix component layer be formed by repeating the alternate placement for two sets or three sets or more. In one or more of embodiments, the combination of the substance A and the substance B is a combination of a protein or polymer having an RGD sequence (hereinafter also referred to as a “substance having an RGD sequence”) and a protein or polymer that interacts with the protein or polymer having an RGD sequence (hereinafter also referred to as a “substance having an interaction”), or a combination of a positively charged protein or polymer (hereinafter also referred to as a “positively charged substance”) and a negatively charged protein or polymer (hereinafter also referred to as a “negatively charged substance”). In one or more of embodiments, the solution A (the solution B) contains a substance A (a substance B) and a solvent or a dispersive medium (hereinafter also referred to simply as a “solvent”). In one or more of embodiments, the amount of the substance A (the substance B) contained in the solution A (the solution B) is 0.0001 to 1% by mass, 0.01 to 0.5% by mass, or 0.02 to 0.1% by mass. In one or more of embodiments, examples of the solvent include aqueous solvents such as water, phosphate buffered saline (PBS), and a buffer solution. In one or more of embodiments, examples of the buffer solution include Tris buffer solutions such as a Tris-HCl buffer solution, a phosphate buffer solution, a HEPES buffer solution, a citric acid-phosphate buffer solution, a glycylglycine-sodium hydroxide buffer solution, a Britton-Robinson buffer solution, and a GTA buffer solution. The pH of the solvent is not particularly limited but is 3 to 11, 6 to 8, or 7.2 to 7.4 in one or more of embodiments.

A production method of the present disclosure includes alternately carrying out the formation of a cell layer and the formation of an extracellular matrix component layer to layer a plurality of the cell layers. The number of the cell layers to be layered is not particularly limited but in terms of allowing them to express the properties and functions that are equivalent to those of a biological tissue of, for example, a human being as much as possible, it is preferably at least five layers, at least six layers, or at least seven layers, but preferably not more than 15 layers, not more than 14 layers, not more than 13 layers, not more than 12 layers, not more than 11 layers, or not more than ten layers. In one or more of embodiments, the first layering method can be carried out by referring to the method disclosed in JP 4919464B.

### Second Layering Method

The second layering method includes layering smooth muscle cells coated with an extracellular matrix component to three-dimensionally layer smooth muscle cells directed towards a differentiated type from an undifferentiated type.

In one or more of embodiments, the smooth muscle cells coated with an extracellular matrix component (hereinafter also referred to as “coated cells”) include smooth muscle cells directed towards a differentiated type from an undifferentiated type and a film containing the extracellular matrix component that coats the smooth muscle cells (hereinafter also referred to as an “extracellular matrix component film”). It is preferable that the extracellular matrix component film include a film containing a substance A and a film containing a substance B that interacts with the substance A. The combinations of the substance A and the substance B are as described above.

In one or more of embodiments, the thickness of the extracellular matrix component film is 1 to 1×10³ nm or 2 to 1×10² nm and is preferably 3 to 1×10² nm because a three-dimensional tissue with coated cells layered more densely can be obtained. The thickness of the extracellular matrix component film can be controlled suitably by, for example, the number of the films that constitute the film. The extracellular matrix component film is not particularly limited but may be of one layer or may be of a multilayer of, for example, 3, 5, 7, 9, 11, 13, or 15 layers or more in one or more of embodiments.

In one or more of embodiments, layering of coated cells includes seeding coated cells in such a manner that the coated cells are in a three-dimensionally layered state and culturing them in a culture medium. In one or more of embodiments, the density of the coated cells at the time of seeding can be determined suitably according to, for example, the size and thickness of the target three-dimensional tissue, the size of the container for culturing, and the number of the cells to be layered. In one or more of embodiments, the density is 1×10² to 1×10⁹ pcs/cm³, 1×10⁴ to 1×10⁸ pcs/cm³, or 1×10⁵ to 1×10⁷ pcs/cm³. The culture medium and the culture conditions are as described above.

In one or more of embodiments, the coated cells can be prepared by alternately bringing a solution containing a substance A (solution A) and a solution containing a substance B (solution B) into contact with smooth muscle cells directed towards a differentiated type from an undifferentiated type. The solution A and the solution B are as described above. In one or more of embodiments, the second layering method can be carried out by referring to the method disclosed in JP 2012-115254 A.

In terms of improving the expression of elastic fibers in a three-dimensional tissue and improving the self-supporting property of the three-dimensional tissue, in one or more of embodiments, the production method of the present disclosure may include culturing a layered product, which was obtained by layering cells, for at least one day. In one or more of embodiments, the period of time for culturing the cells is at least two days, at least three days, at least four days, at least five days, at least six days, at least seven days, at least ten days, or at least 15 days but not more than 30 days, not more than 25 days, or not more than 21 days.

In the production method of the present disclosure, in terms of allowing the properties and/or functions that are equivalent to those of a biological tissue of, for example, a human being as much as possible to be expressed, in one or more of embodiments, it is preferable that a cell suspension containing vascular endothelial cells be placed on the cell layer with smooth muscle cells layered and then be cultured. In one or more of embodiments, it is preferable that the cell suspension be placed in such a manner that one cell layer of the vascular endothelial cells is obtained. The culture conditions are as described above.

In the production method of the present disclosure, in terms of allowing the properties and/or functions that are equivalent to those of a biological tissue of, for example, a human being as much as possible to be expressed, in one or more of embodiments, it is preferable that the above-mentioned cell suspension containing smooth muscle cells be placed on a fibroblast layer including fibroblasts layered with an extracellular matrix component interposed therebetween to form a cell layer with the smooth muscle cells being layered.

### Vascular Prosthesis

In one or more of embodiments, the present disclosure relates to a vascular prosthesis obtained by shaping a three-dimensional tissue of the present disclosure. Since the vascular prosthesis of the present disclosure is obtained by shaping the three-dimensional tissue of the present disclosure, it is excellent in self-supporting property in one or more of embodiments. In one or more of embodiments, the shape of the vascular prosthesis of the present disclosure is preferably tubular.

### Evaluation Method

In one or more of embodiments, the present disclosure relates to a method of evaluating the effect of a test material on a blood vessel using a three-dimensional tissue of the present disclosure. According to the evaluation method of the present disclosure, in one or more of embodiments, the test material can be evaluated in an environment similar to that of an actual blood vessel. The evaluation method of the present disclosure can be a very useful tool for pharmacokinetic evaluation of drugs of various molecular weights in, for example, creating (screening) new drugs.

In one or more of embodiments, the evaluation method of the present disclosure includes contacting a test material to the three-dimensional tissue of the present disclosure, observing the effect of the test material on the three-dimensional tissue, and evaluating the test material based on the observations.

### Evaluation Kit

In one or more of embodiments, the present disclosure relates to an evaluation kit for a test material. The kit of the present disclosure includes a three-dimensional tissue of the present disclosure. In one or more of embodiments, the kit of the present disclosure may further include a product containing at least one selected from a reagent, a material, a tool, and a device that are used for a predetermined test as well as the instructions (an instruction manual) about the evaluation thereof.

Hereinafter, the substance having an RGD sequence, the substance having an interaction, the positively charged substance, and the negatively charged substance, which were described as extracellular matrix components, are described by examples.

### Substance Having RGD Sequence

The substance having an RGD sequence denotes a protein or polymer that has an “Arg-Gly-Asp” (RGD) sequence that is an amino acid sequence responsible for a cell adhesion activity. In the present specification, “having an RGD sequence” may denote originally having an RGD sequence or having an RGD sequence bonded chemically. It is preferable that the substance having an RGD sequence be biodegradable.

In one or more of embodiments, examples of the protein having an RGD sequence include conventionally known adhesion proteins and water-soluble proteins having an RGD sequence. In one or more of embodiments, examples of the adhesion proteins include fibronectin, vitronectin, laminin, cadherin, and collagen. In one or more of embodiments, examples of the water-soluble proteins having an RGD sequence include collagen, gelatin, albumin, globulin, proteoglycan, enzymes, and antibodies, to which an RGD sequence was bonded.

In one or more of embodiments, examples of the polymer having an RGD sequence include naturally occurring polymers and synthetic polymers. In one or more of embodiments, examples of the naturally occurring polymers having an RGD sequence include water-soluble polypeptides, low molecular weight peptides, poly amino acids such as a-polylysine and g-polylysine, as well as sugars such as chitin and chitosan. In one or more of embodiments, examples of the synthetic polymers having an RGD sequence include polymers or copolymers having an RGD sequence with, for example, a linear, graft, comb, dendritic, or star structure. In one or more of embodiments, examples of the polymers or copolymers include polyurethane, polycarbonate, polyamide, and copolymers thereof, polyester, poly(N-isopropyl acrylamide-co-polyacrylic acid), polyamideamine dendiimer, polyethylene oxide, poly(ε-caprolactam), polyacrylamide, and poly (methyl methacrylate-γ-polyoxyethylene methacrylate).

Among these, the substance having an RGD sequence is preferably fibronectin, vitronectin, laminin, cadherin, polylysine, elastin, collagen with an RGD sequence bonded thereto, gelatin with an RGD sequence bonded thereto, chitin, or chitosan, more preferably fibronectin, vitronectin, laminin, polylysine, collagen with an RGD sequence bonded thereto, or gelatin with an RGD sequence bonded thereto.

### Interacting Substance

The interacting substance denotes a protein or polymer that interacts with a substance having an RGD sequence. In the present specification, “interacting” denotes that in one or more of embodiments, a substance having an RGD sequence and an interacting substance approach to each other to the extent that bonding, adhesion, adsorption, or electron transfer can occur chemically and/or physically between the substance having an RGD sequence and the interacting substance through, for example, electrostatic interaction, hydrophobic interaction, hydrogen bond, charge transfer interaction, covalent bonding, specific interaction between proteins, and/or van der Waals' force. The interacting substance is preferably biodegradable.

In one or more of embodiments, examples of the protein that interacts with the substance having an RGD sequence include collagen, gelatin, proteoglycan, integrin, enzymes, and antibodies. In one or more of embodiments, examples of the polymer that interacts with the substance having an RGD sequence include naturally occurring polymers and synthetic polymers. In one or more of embodiments, examples of the naturally occurring polymers that interact with the substance having an RGD sequence include water-soluble polypeptides, low molecular weight peptides, polyamino acids, elastin, sugars such as heparin, heparan sulfate, and dextran sulfate, and hyaluronic acids. In one or more of embodiments, examples of polyamino acids include polylysine such as α-polylysine and ε-polylysine, polyglutamic acid, and polyaspartic acid. In one or more of embodiments, examples of the synthetic polymers that interact with the substance having an RGD sequence include those described as examples of the above-mentioned synthetic polymers having an RGD sequence.

Among these, the interacting substance is preferably gelatin, dextran sulfate, heparin, hyaluronic acid, globulin, albumin, polyglutamic acid, collagen, or elastin, more preferably gelatin, dextran sulfate, heparin, hyaluronic acid, or collagen, and further preferably gelatin, dextran sulfate, heparin, or hyaluronic acid.

The combination of the substance having an RGD sequence and the interacting substance is not particularly limited as long as it is a combination of different substances that interact with each other and as long as one of them is a polymer or protein including an RGD sequence while the other is a polymer or protein that interacts therewith. In one or more of embodiments, examples of the combination of the substance having an RGD sequence and the substance having an interaction include combinations of fibronectin and gelatin, fibronectin and ε-polylysine, fibronectin and hyaluronic acid, fibronectin and dextran sulfate, fibronectin and heparin, fibronectin and collagen, laminin and gelatin, laminin and collagen, polylysine and elastin, vitronectin and collagen, and RGD-bonded collagen or RGD-bonded gelatin and collagen or gelatin. Among these, the combination of fibronectin and gelatin, fibronectin and ε-polylysine, fibronectin and hyaluronic acid, fibronectin and dextran sulfate, fibronectin and heparin, or laminin and gelatin is preferable, and the combination of fibronectin and gelatin is more preferable. One type of each of the substance having an RGD sequence and the substance having an interaction may be used, or two or more types of each of them may be used together as long as they interact with each other.

### Positively Charged Substance

The positively charged substance denotes a positively charged protein or polymer. In one or more of embodiments, the positively charged protein is preferably a water-soluble protein. In one or more of embodiments, examples of the water-soluble protein include basic collagen, basic gelatin, lysozyme, cytochrome c, peroxidase, and myoglobin. In one or more of embodiments, examples of the positively charged polymer include naturally occurring polymers and synthetic polymers. In one or more of embodiments, examples of the naturally occurring polymers include water-soluble polypeptides, low molecular weight peptides, poly amino acids, and sugars such as chitin and chitosan. In one or more of embodiments, examples of the poly amino acids include polylysine such as poly(α-lysine) and poly(ε-lysine), polyarginine, and polyhistidine. In one or more of embodiments, examples of the synthetic polymers include polymers and copolymers with, fir example, a linear, graft, comb, dendritic, or star structure. In one or more of embodiments, examples of the polymers and copolymers include polyurethane, polyamide, polycarbonate, and copolymers thereof, polyester, polydiallyldimethylammonium chloride (PDDA), polyallylamine hydrochloride, polyethyleneimine, polyvinylamine, and polyamideamine dendrimer.

### Negatively Charged Substance

The negatively charged substance denotes a negatively charged protein or polymer. In one or more of embodiments, the negatively charged protein is preferably a water-soluble protein. In one or more of embodiments, examples of the water-soluble protein include acidic collagen, acidic gelatin, albumin, globulin, catalase, β-lactoglobulin, thyroglobulin, α-lactalbumin, and ovalbumin. Examples of the negatively charged polymer include naturally occurring polymers and synthetic polymers. In one or more of embodiments, examples of the naturally occurring polymers include water-soluble polypeptides, low molecular weight peptides, polyamino acids such as poly(β-lysine), and dextran sulfate. In one or more of embodiments, examples of the synthetic polymers include polymers and copolymers with, for example, a linear, graft, comb, dendritic, or star structure. In one or more of embodiments, examples of the polymers and copolymers include polyurethane, polyamide, polycarbonate, and copolymers thereof, polyester, polyacrylic acid, polymethacrylic acid, polystyrene sulfonate, polyacrylamidomethylpropane sulfonic acid, terminal-carboxylated polyethylene glycol, polydiallyldimethylammonium salt, polyallylamine salt, polyethyleneimine, polyvinylamine, and polyamideamine dendrimer.

In one or more of embodiments, examples of the combination of the positively charged substance and the negatively charged substance include combinations of ε-polylysine salt and polysulfonate, ε-polylysine and polysulfonate, chitosan and dextran sulfate, polyallylamine hydrochloride and polystyrene sulfonate, polydiallyldimethylammonium chloride and polystyrene sulfonate, and polydiallyldimethylammonium chloride and polyacrylate. Preferably, the combination is a combination of ε-polylysine salt and polysulfonate or polydiallyldimethylammonium chloride and polyacrylate. In one or more of embodiments, examples of polysulfonate include sodium polysulphonate (PSS). One type of each of the positively charged substance and the negatively charged substance may be used, or two or more types of each of them may be used together as long as they interact with each other.

The present disclosure may relate to one or more of the following embodiments.

<1> An elastic three-dimensional tissue, including smooth muscle cells and an extracellular matrix component, with the smooth muscle cells being layered with the extracellular matrix component interposed therebetween.

<2> The three-dimensional tissue according to the item <1>, wherein the three-dimensional tissue can be elongated to at least 1.2 times its length.

<3> A production method for a three-dimensional tissue, including layering smooth muscle cells with an extracellular matrix component interposed therebetween, wherein the smooth muscle cells are those directed towards a differentiated type from an undifferentiated type.

<4> The production method according to the item <3>, wherein the method includes culturing smooth muscle cells at a high density to produce the smooth muscle cells.

<5> The production method according to the item <3> or <4>, wherein the smooth muscle cells are those of a fetal or infancy stage.

<6>The production method according to any one of items <3> to <5>, wherein the layering includes alternately carrying out the formation of a cell layer of the smooth muscle cells and the formation of a layer containing the extracellular matrix component or layering smooth muscle cells coated with the extracellular matrix component.

<7> An elastic three-dimensional tissue, produced by a production method according to any one of the items <3> to <6>.

Hereinafter, the present disclosure is further described by way of examples and comparative examples. However, the present disclosure is not interpreted to be limited to the following examples.

### EXAMPLES

### Example 1

### Preparation of Smooth Muscle Cell (SMC) Solution

Aorta smooth muscle cells recovered from a rat neonate were subcultured four times, which then were cultured for 11 days. In the 11-day culture, they were cultured at a density of at least 95% confluent for seven days. The cells that were trypsinized (0.05% trypsin, 0.02% EDTA) (37° C., 5 to 7 minutes) and then were recovered were seeded at a density of 50% confluent to be cultured for five days. In the five-day culture, the proliferative capacity of the cells was extremely low. Therefore, it was able to be confirmed that the cells were smooth muscle cells directed towards a differentiated type from an undifferentiated type. The cells cultured for five days were trypsinized under the same conditions as described above and then were recovered. Thereafter; they were dispersed in a culture medium to have a density of 4.0×10⁴ cells/mL. Thus, an SMC solution was prepared. In this case, the culture medium used herein was DMEM (Dulbecco's Modified Eagle Medium) containing 10% fetal bovine serum (FBS) and the culture medium was replaced every 48 hours.

### Preparation of Fibronectin Solution (BFN Solution)

Bovine plasma-derived fibronectin (Product No. F1141, manufactured by SIGMA, solution, 1 mg/mL (0.5M NaCl, 0.05M Tris (pH 7.5)) was diluted with 0.5M NaCl, 0.05M Tris (pH 7.5) to be 0.2 mg/mL. Thus, a BFN solution was prepared.

### Preparation of Gelatin Solution

Gelatin (Product No. 077-03155, manufactured by Wako) was dissolved in 0.05M Tris (pH 7.5) at 37° C. over 3 to 4 hours to be 0.2 mg/ml. Thus, a gelatin solution was prepared.

### Production of Three-Dimensional Tissue

A cell disk (Product Name: Cell Disk LF; manufactured by Sumitomo Bakelite) was immersed in 2 ml of BFN solution (37° C., 1 min each) and thereby a BFN layer was formed on the surface of the cell disk. Thereafter; the SMC solution was placed on the BFN layer. The SMC solution was placed in such a manner that the SMC were seeded at 11×10⁴ cells/cm². This then was cultured in a cell culture incubator (37T, 5% CO₂) for half a day to allow the cells to adhere thereto. Thus, an SMC layer (a first layer) was formed. Subsequently, the SMC layer was immersed alternately in 2 mL of BFN solution and 2 mL of gelatin solution for a total of nine times (37° C., 1 min each) and thereby a fibronectin gelatin (FN-G) nano thin film was formed on the surface of the SMC layer. The SMC solution was placed on the FN-G nano thin film immediately (SMC: 11×10⁴ cells/cm²). This then was cultured in the cell culture incubator (37° C., 5% CO₂) for 6 to 12 hours to allow the cells to adhere thereto. Thus, an SMC layer (a second layer) was formed. The formation of the SMC layer and the formation of the FN-G nano thin film were alternately carried out repeatedly and thereby seven SMC layers were layered in four days. Thereafter, they were cultured for three days and thereby a three-dimensional tissue including seven SMC layers was formed. In this case, with respect to the culture media, DMEM containing 10% FBS was used while the seven SMC layers were layered, and for the three days thereafter, DMEM containing 1 to 2% FBS was used. The culture medium was replaced daily.

### Comparative Example 1

### Preparation of Cell Suspension

Aorta smooth muscle cells recovered from a rat neonate were subcultured for five to seven times, which then were trypsinized (0.05% trypsin, 0.02% EDTA) (37° C., 5 to 7 minutes) at a density of 80% confluent. Thereafter, the cells were recovered. The cell thus recovered were dispersed in a culture medium (DMEM containing 10% FBS) to have a density of 4.0×10⁴ cells/mL. Thus, a cell suspension was prepared. In this case, the culture medium was replaced every 48 hours.

### Production of Three-Dimensional Tissue

Seven SMC layers were layered in four days in the same procedure as in Example 1 except for using the above-mentioned cell suspension instead of the SMC solution. Thereafter, they were cultured in DMEM containing 1-2% FBS at 37° C. for 48 hours. Thus, a three-dimensional tissue including the seven SMC layers was formed.

### Evaluation by Tissue Staining

Expressions of the elastic fibers of the resultant three-dimensional tissues and the blood vessels of rats were evaluated by Elastica van Gieson stain. The images thereof are shown in FIGS. 1A to 1D. FIG. 1A is an image of the three-dimensional tissue of Example 1, FIG. 1B is an image of the blood vessel of the rat neonate, FIG. 1C is an image of the blood vessel of an adult rat, and FIG. 1D is an image of the three-dimensional tissue of Comparative Example 1.

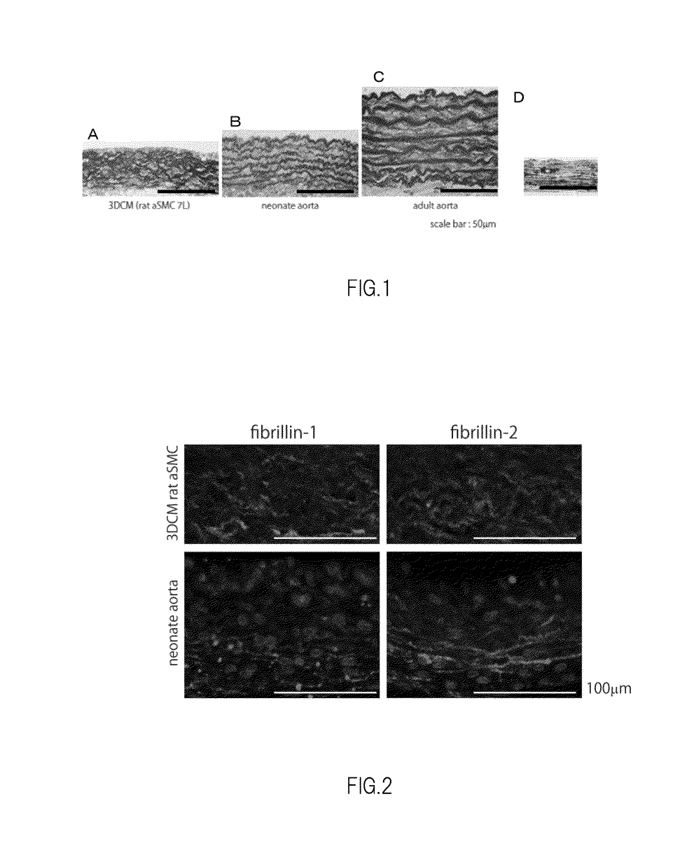

Furthermore, expressions of fibrillin-1 and -2 that are important for the expression of elastic fibers were evaluated by immunostaining. The images thereof are shown in FIG. 2. FIG. 2 shows examples of photographs of fluorescent immunostaining tissue sections of the resultant three-dimensional tissue. The upper images are images of the three-dimensional tissue of Example 1, while the lower images are images of the blood vessel of the rat neonate.

As shown in FIGS. 1A and 1D, the three-dimensional tissue of Example 1 had an extremely high level of expression of elastic fibers as compared to the three-dimensional tissue of Comparative Example 1. As shown in FIGS. 1A to 1C and FIG. 2, it was observed that the three-dimensional tissue of Example 1 had a high level of expression of elastic fibers, which was comparable to those of the rat neonate and adult rat. Furthermore, the elastic fibers of the three-dimensional tissue of Example 1 are in the form of thick layer as is the case with the elastic fibers of the rat neonate and adult rat. These suggested that the three-dimensional tissue of Example 1 was highly elastic.

### Visual Evaluation of Elasticity

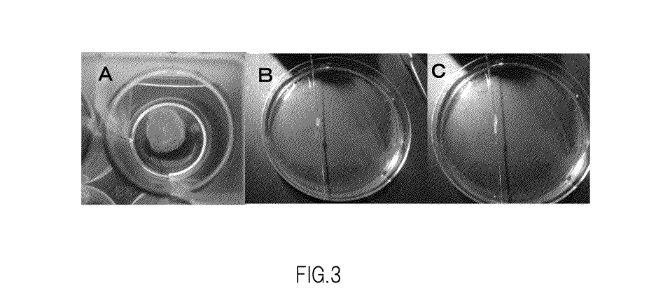

The elasticity of the three-dimensional tissue produced in Example 1 was evaluated visually. The images of this evaluation experiment are shown in FIG. 3. That is, FIG. 3 shows, sequentially from the left, an image of the three-dimensional tissue produced in Example 1 dissociated from the cell disk, an image of the three-dimensional tissue dissociated, which was wound on a capillary, and an image of the appearance thereof pulled longitudinally, respectively. As shown in FIG. 3, the three-dimensional tissue of Example 1 had a self-supporting property to the extent that it can be formed into a tubular shape, and it was elongated to approximately twice its length with respect to the longitudinal direction (the direction of elongation). Therefore, it was confirmed that the three-dimensional tissue of Example 1 had a sufficient self-supporting property as well as excellent elasticity. The three-dimensional tissue of Comparative Example 1 was not able to be pulled.

Thus, the method of Example 1 made it possible to produce a three-dimensional tissue with excellent elasticity in a short period of time.

### Comparative Example 2

A three-dimensional tissue was produced in the same manner as in Example 1 except that in the preparation of the cell suspension, the period of time for culturing the cells subcultured four times was nine days (culturing at a density of at least 95% confluent: five days) instead of 11 days. The smooth muscle cells used for layering had a high cell proliferative capacity and had not been differentiated into a differentiated type from an undifferentiated type. The smooth muscle cells were able to be layered but the resultant three-dimensional tissue had no elasticity.

### Example 2

### Production of Three-Dimensional Tissue

The formation of an SMC layer and the formation of an FN-G nano thin film were alternately carried out repeatedly in the same manner as in Example 1 except that one SMC layer was layered per day that is, seven SMC layers were layered in seven days (the culture period after placing the SMC solution was 12 to 24 hours). Thereafter, they were cultured for three days and thereby a three-dimensional tissue including the seven SMC layers was formed.

With respect to the three-dimensional tissue thus obtained, the elasticity thereof was evaluated visually. The three-dimensional tissue produced was dissociated from the cell disk, which then was pulled longitudinally. As a result, it was elongated to approximately twice its length in the direction of elongation.

1-7. (canceled)

8. An elastic three-dimensional tissue, comprising smooth muscle cells and an extracellular matrix component, with the smooth muscle cells being layered with the extracellular matrix component interposed therebetween.

9. The three-dimensional tissue according to claim 8, wherein the three-dimensional tissue can be elongated to at least 1.2 times its length.

10. A production method for a three-dimensional tissue, comprising layering smooth muscle cells with an extracellular matrix component interposed therebetween,
wherein the smooth muscle cells are those directed towards a differentiated type from an undifferentiated type.

11. The production method according to claim 10, wherein the method comprises culturing smooth muscle cells at a high density to produce the smooth muscle cells.

12. The production method according to claim 10, wherein the smooth muscle cells are those of a fetal or infancy stage.

13. The production method according to claim 10, wherein the layering comprises alternately carrying out formation of a cell layer of the smooth muscle cells and formation of a layer containing the extracellular matrix component or layering smooth muscle cells coated with the extracellular matrix component.

14. An elastic three-dimensional tissue, produced by a production method according to claim 10.

15. A production method for a three-dimensional tissue, comprising:
culturing smooth muscle cells in a substantially 100% confluent state;
dissociating the cells thus cultured to recover them; and
layering the cells thus recovered, with an extracellular matrix component being interposed therebetween, and then culturing them.

16. The production method according to claim 15, wherein the culturing the smooth muscle cells includes culturing smooth muscle cells selected from the group consisting of smooth muscle progenitor cells, smooth muscle cells of an undifferentiated type, and smooth muscle cells of a dedifferentiated type until they become substantially 100% confluent, and further culturing them in a substantially 100% confluent state.

17. The production method according to claim 15, wherein the smooth muscle cells are cultured in the substantially 100% confluent state and thereby the smooth muscle cells are made to be smooth muscle cells directed towards a differentiated type from an undifferentiated type.

18. The production method according to claim 16, wherein the smooth muscle cells are cultured in the substantially 100% confluent state and thereby the smooth muscle cells are made to be smooth muscle cells directed towards a differentiated type from an undifferentiated type.

19. The production method according to claim 15, wherein the layering comprises alternately carrying out formation of a cell layer of the smooth muscle cells and formation of a layer containing the extracellular matrix component or layering smooth muscle cells coated with the extracellular matrix component.

20. The production method according to claim 15, wherein the smooth muscle cells are those of a fetal or infancy stage.

21. The production method according to claim 8, wherein the method comprises culturing smooth muscle cells in a substantially 100% confluent state.

22. The production method according to claim 21, wherein the culturing the smooth muscle cells includes further culturing them in a substantially 100% confluent state.

23. New The production method according to claim 20, wherein the smooth muscle cells is selected from the group consisting of smooth muscle progenitor cells, smooth muscle cells of an undifferentiated type, and smooth muscle cells of a dedifferentiated type.

24. The production method according to claim 21, wherein the method comprises dissociating the cells thus cultured to recover them; and
wherein the layering smooth muscle cells including layering the cells thus recovered, with an extracellular matrix component being interposed therebetween, and then culturing them.

25. The production method according to claim 20, wherein the culturing the smooth muscle cells includes culturing smooth muscle cells selected from the group consisting of smooth muscle progenitor cells, smooth muscle cells of an undifferentiated type, and smooth muscle cells of a dedifferentiated type until they become substantially 100% confluent, and further culturing them in a substantially 100% confluent state.
